SEQUENCE LISTING (4 sequences)

SEQ ID NO: 1 - DNA, 37 bp, Artificial Sequence
  Description of Artificial Sequence Forward Primer A
tcattcgcaatcagcttgaaggaattcttgaaaacga

SEQ ID NO: 2 - DNA, 33 bp, Artificial Sequence
  Description of Artificial Sequence Forward Primer B
tcattcgcaatcaatgatttccctgaccttggc

SEQ ID NO: 3 - DNA, 27 bp, Artificial Sequence
  Description of Artificial Sequence Reverse Primer
accttaggtgtccttctaaggatcctg

SEQ ID NO: 4 - DNA, 13 bp, Artificial Sequence
  Description of Combined DNA/RNA Molecule Common Annealing Polymer
tgattgcgaatga

(19) United States
(12) Patent Application Publication
(10) Pub. No.: US 2005/0123975 A1
(43) Pub. Date: Jun. 9, 2005

(54) Methods for determining the degradation state or concentration of nucleic acids

(75) Inventors: Lori K. Hennessy, San Mateo, CA (US); Jens J. Hyldig-Nielsen, Holliston, MA (US)

Correspondence Address: Applied Biosystems, 35 Wiggins Avenue, BEDFORD, MA, 01730, US

(73) Assignee: Applera Corporation, Bedford, MA (US)

(21) Appl. No.: 10/993,960

(22) Filed: Nov. 19, 2004

Related U.S. Application Data
(60) Provisional application No. 60/523,526, filed on Nov. 19, 2003.

(51) Int. Cl. C12Q 1/68; C12P 19/34; C07K 14/00

(52) U.S. Cl. 435/6; 435/91.2; 530/350

(57) ABSTRACT

Embodiments of this invention are directed to methods for determining or estimating the concentration of nucleic acid or the state of degradation of nucleic acid in a sample of interest.

### CROSS REFERENCE TO RELATED APPLICATIONS

This application claims the benefit of U.S. Provisional Application No. 60/523,526, filed on Nov. 19, 2003, incorporated herein by reference.

### TECHNICAL FIELD

This invention pertains to the field of nucleic acid analysis.

### BRIEF DESCRIPTION OF THE DRAWINGS

FIG. 1 is an illustration of an embodiment of a primer complex.

FIG. 2A is an illustration of a plasmid comprising priming sites for two primer complexes.

FIG. 2B is an illustration of an expanded view of a section on FIG. 2A wherein the forward primer complex has hybridized to a primer site of the plasmid and is being extended by a polymerase enzyme.

FIG. 3 is an illustration of the process and possible products produced by operation of the first round of PCR using primer complexes as primers in a PCR reaction.

FIG. 4 is an illustration of the process and possible products produced by operation of the second round of PCR using primer complexes as primers in a PCR reaction.

FIG. 5 is an illustration of possible PCR products produced by the operation of several cycles of PCR using primer complexes as primers in a PCR reaction.

### DESCRIPTION OF THE INVENTION

1. List of Certain Abbreviations Used Herein:

Fmoc=9-fluorenylmethoxycarbonyl
Bhoc=benzhydroloxycarbonyl
PAL=5-(4′-aminomethyl-3′,5′-dimethoxyphenoxy)valeric acid
MBHA=methylbenzhydrylamine
PNA=peptide nucleic acid
XAL=Xanthenylamide
PCR=polymerase chain reaction
VIC=fluorescent dye available as a label on oligomers ordered from Applied Biosystems
FAM or 6-FAM=fluorescent dye available as a label on oligomers ordered from Applied Biosystems
dabcyl=4-((4-(dimethylamino)phenyl)azo)benzoic acid
   2. Definitions:

For the purposes of interpreting this specification the following definitions shall apply and whenever appropriate, terms used in the singular shall also include the plural and vice versa.

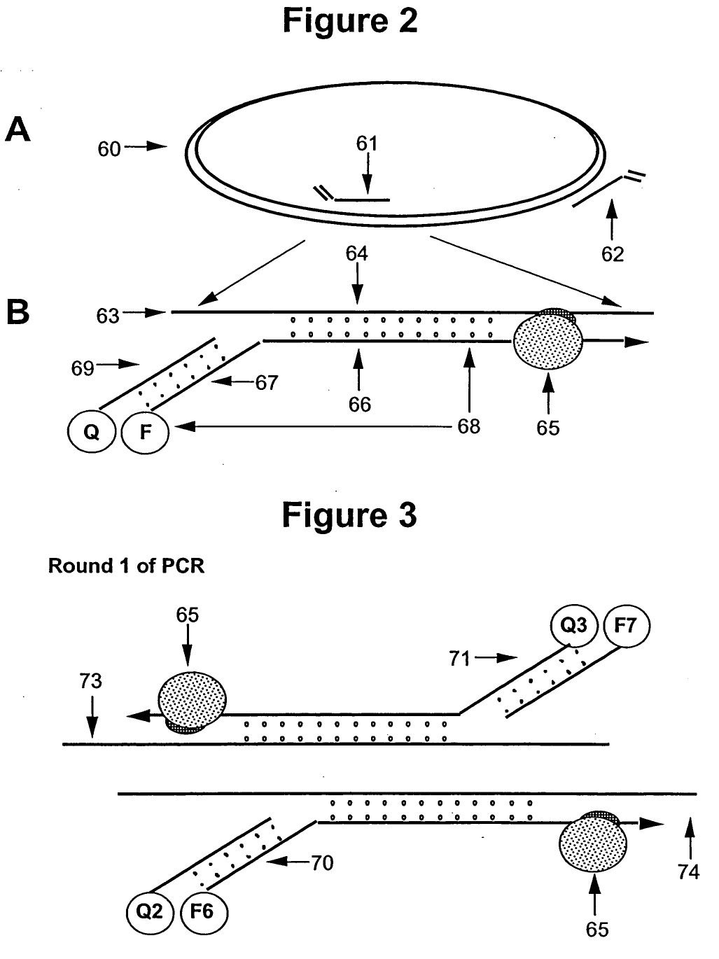

a. As used herein, “nucleobase” refers to those naturally occurring and those non-naturally occurring heterocyclic moieties commonly known to those who utilize nucleic acid technology or utilize peptide nucleic acid technology to thereby generate polymers that can sequence specifically bind to nucleic acids. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothyrnine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases illustrated in FIGS. 2(A) and 2(B) of Buchardt et al. (U.S. Pat. No. 6,357,163, herein incorporated by reference).

b. As used herein, “nucleobase polymer” refers to a polymer comprising a series of linked nucleobase containing subunits. Non-limiting examples of suitable polymers include oligodeoxynucleotides, oligoribonucleotides, peptide nucleic acids, nucleic acid analogs, nucleic acid mimics and chimeras.

c. As used herein, “nucleobase sequence” refers to nucleobase polymer, or a segment of a nucleobase polymer.

d. As used herein, “primer sequence” or “primer site” refers to a nucleobase sequence of a target nucleic acid molecule to which a primer or primer complex is designed to anneal for the purpose of initiating a polymerase extension reaction.

e. As used herein, “target nucleic acid molecule” refers to a nucleic acid molecule of interest. The target nucleic acid molecule can be double stranded or single stranded. Moreover, a sample can comprise more than one target nucleic acid molecule.

f. As used herein, “amplified molecule” refers to a nucleic acid molecule that is produced by operation of an amplification process.

g. As used herein, “non-nucleic acid polymer” means a nucleobase polymer that does not comprise nucleotides. One example of a non-nucleic acid polymer is a peptide nucleic acid (PNA) polymer.

h. As used herein, “peptide nucleic acid” or “PNA” refers to any oligomer or polymer comprising two or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference. The term “peptide nucleic acid” or “PNA” can also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

In some embodiments, a “peptide nucleic acid” or “PNA” is an oligomer or polymer segment comprising two or more covalently linked subunits of the formula: 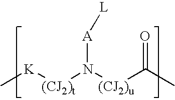
wherein, each J is the same or different and is selected from the group consisting of H, R¹, OR¹, SR¹, NHR¹, NR², F, Cl, Br and I. Each K is the same or different and is selected from the group consisting of O, S, NH and NR¹. Each R¹ is the same or different and is an alkyl group having one to five carbon atoms that can optionally contain a heteroatom or a substituted or unsubstituted aryl group. Each A is selected from the group consisting of a single bond, a group of the formula; —(CJ₂)ₛ- and a group of the formula; —(CJ₂)ₛC(O)—, wherein, J is defined above and each s is a whole number from one to five. Each t is 1 or 2 and each u is 1 or 2. Each L is the same or different and is independently selected from: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine), other naturally occurring nucleobase analogs or other non-naturally occurring nucleobases.

i. As used herein, the terms “label” and “detectable moiety” are interchangeable and refer to moieties that can be attached to a nucleobase polymer to thereby render the nucleobase polymer detectable by an instrument or method.

j. As used herein, “chimera” or “chimeric oligomer” refers to an oligomer or polymer comprising two or more linked subunits that are selected from different classes of subunits. For example, a PNA/DNA chimera would comprise at least two PNA subunits linked to at least one 2′-deoxyribonucleic acid subunit (For exemplary methods and compositions related to PNA/DNA chimera preparation See: WO96/40709). Exemplary component subunits of the chimera are selected from the group consisting of PNA subunits, naturally occurring amino acid subunits, DNA subunits, RNA subunits and subunits of analogues or mimics of nucleic acids.

k. As used herein “component polymer” or “component polymers” refers to the two or more nucleobase polymers that assemble to form a primer complex.

m. As used herein, “support”, “solid support” or “solid carrier” refers to any solid phase material. Solid support encompasses terms such as “resin”, “synthesis support”, “solid phase”, “surface” “membrane” and/or “support”. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled-pore-glass (CPG), or reverse-phase silica. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression or other container, vessel, feature or location.

3. General Description Relevant to the Various Embodiments of the Invention:

PNA Oligomer Synthesis Through Chemical Assembly:

Methods for the chemical assembly of PNAs are well-known (See: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,201,103, 6,228,982 and 6,357,163; all of which are herein incorporated by reference (Also see: PerSeptive Biosystems Product Literature)). As a general reference for PNA synthesis methodology also please see: Nielsen et al., Peptide Nucleic Acids; Protocols and Applications, Horizon Scientific Press, Norfolk England (1999).

Chemicals and instrumentation for the support bound automated chemical assembly of peptide nucleic acids are now commercially available. Both labeled and unlabeled PNA oligomers are likewise available from commercial vendors of custom PNA oligomers (e.g. Applied Biosystems). Chemical assembly of a PNA is analogous to solid phase peptide synthesis, wherein at each cycle of assembly the oligomer possesses a reactive alkyl amino terminus that is condensed with the next synthon to be added to the growing polymer.

PNA can be synthesized at any scale, from submicromole to millimole, or more. PNA can be conveniently synthesized at the 2 μmole scale, using Fmoc(Bhoc), tBoc/Z, or MmT protecting group monomers on an Expedite Synthesizer (Applied Biosystems) using a XAL or PAL support. Alternatively the Model 433A Synthesizer (Applied Biosystems) with MBHA support can be used. Moreover, many other automated synthesizers and synthesis supports can be utilized. Because standard peptide chemistry can be utilized, natural and non-natural amino acids can be routinely incorporated into a PNA oligomer. Because a PNA is a polyamide, it has a C-terminus (carboxyl terminus) and an N-terminus (amino terminus). For the purposes of the design of a priming segment suitable for antiparallel binding to priming site (the preferred orientation), the N-terminus of the priming nucleobase sequence of the PNA polymer is the equivalent of the 5′-hydroxyl terminus of an equivalent DNA or RNA oligonucleotide.

PNA Oligomer Synthesis Through Ligation/Condensation

PNA oligomers can also be prepared by ligation in the absence of a template as described in more detail in WIPO application number WO02/72865 as well as in copending and commonly owned patent application numbers U.S. Ser. No. 10/096,125, U.S. Ser. Nos. 10/655,731 and 10/696,016, all of which are herein incorporated by reference. Generally, when used in ligation/condensation reactions, the nature of the ligation chemistry chosen should be considered. For simplicity, we sometimes refer to one of the oligomers used in a ligation/condensation reaction as a terminal oligomer or terminal block and the other as the condensation oligomer or condensation block. This distinction is generally irrelevant except to distinguish between the different blocks especially if they contain the same nucleobase sequence. Often, at least the nature of the functional groups that are used in the ligation can be different for the terminal and condensation oligomer blocks since they can be designed to accommodate different ligation chemistries. For example, one of the oligomer blocks can comprise a C-terminal acid group and the other can comprise an N-terminal amine group wherein the product of the condensation/ligation reaction is an amide bond that forms an elongated PNA oligomer or chimera.

However, when the oligomer is to be extended by multiple ligations, we will generally refer to the terminal oligomer block as the oligomer block produced from the first ligation or from the immediately preceding ligation step.

The terminal blocks can comprise a C-terminal amide that is relatively unreactive. In contrast, the condensing blocks can comprise a C-terminal end comprising a functional group that is suitable for performing the ligation reaction. For example, depending upon the nature of the condensation chemistry, the C-terminal end of the oligomer can comprise a C-terminal acid functional group. If a functional group, the termini of an oligomer to be ligated/condensed may or may not require the addition of a terminal protecting group depending on the nature of the condensation/ligation chemistry. Since the oligomer blocks are themselves often prepared by de novo methods and because suitable commercial reagents and instrumentation are available for the production of PNA oligomers comprising a C-terminal amino acid or C-terminal amide, one of skill in the art can easily prepare the oligomer blocks of the desired C-terminal configuration.

With respect to the N-terminus, again the exact configuration can depend on the nature of the ligation chemistry chosen and on whether or not the oligomer is a condensing oligomer block or a terminal oligomer block. If the oligomer is a terminal block, the N-terminus can comprise a reactive functional group (e.g. N-terminal amine group) whereas if the oligomer is a condensing oligomer block, the N-terminus can be capped. Non-limiting examples of capping include labeling the N-terminus with a label or otherwise reacting it with a relatively non-reactive moiety such as acetyl. In other embodiments, the N-terminus can be capped or protected with a protecting group. If the N-terminus is to be involved in the ligation reaction, it will typically exist as a free amine but may be transiently protected with a protecting group. Since the oligomer blocks are themselves prepared by de novo methods and because suitable commercial reagents and instrumentation are available for the production of PNA oligomers, one of skill in the art can easily prepare the oligomer blocks of the desired N-terminal configuration.

In addition to modification of the termini for ligation, the oligomer blocks can be modified and/or properly protected to thereby incorporate functional groups for labeling or for attachment to surfaces. Such functional groups can be utilized either before or after ligation depending upon factors such as: 1) the oligomer synthesis chemistry (e.g. harsh deprotection conditions required that might destroy a label), the condensation/ligation chemistry chosen (e.g. functional groups of the desired label might interfere with the condensation chemistry) and the intended use of the functional group (e.g. whether it is intended for labeling or for attachment to a solid support).

PNA Labeling/Modification:

Non-limiting methods for labeling PNAs are described in U.S. Pat. No. 6,110,676, U.S. Pat. No. 6,280,964, U.S. Pat. No. 6,355,421, U.S. Pat. No. 6,361,942, and U.S. Pat. No. 6,441,152 and U.S. Pat. No. 6,485,901 (all of which are herein incorporated by reference), or are otherwise well-known in the art of PNA synthesis and peptide synthesis. Methods for labeling PNA are also discussed in Nielsen et al., Peptide Nucleic Acids; Protocols and Applications, Horizon Scientific Press, Norfolk, England (1999). Other non-limiting methods for labeling PNA oligomers are discussed below.

Because the synthetic chemistry of assembly is essentially the same, any method commonly used to label a peptide can often be adapted to effect the labeling of a PNA oligomer. Generally, the N-terminus of the oligomer or polymer can be labeled by reaction with a moiety having a carboxylic acid group or activated carboxylic acid group. One or more spacer moieties can optionally be introduced between the labeling moiety and the nucleobase containing subunits of the oligomer. Generally, the spacer moiety can be incorporated prior to performing the labeling reaction. If desired, the spacer can be embedded within the label and thereby be incorporated during the labeling reaction.

Typically the C-terminal end of the polymer can be labeled by first condensing a labeled moiety or functional group moiety with the support upon which the PNA oligomer is to be assembled. Next, the first nucleobase containing synthon of the PNA oligomer can be condensed with the labeled moiety or functional group moiety. Alternatively, one or more spacer moieties (e.g. 8-amino-3,6-dioxaoctanoic acid; the “O-linker”) can be introduced between the label moiety or functional group moiety and the first nucleobase subunit of the oligomer. Once the molecule to be prepared is completely assembled, labeled and/or modified, it can be cleaved from the support deprotected and purified using standard methodologies.

For example, the labeled moiety or functional group moiety can be a lysine derivative wherein the ε-amino group is a protected or unprotected functional group or is otherwise modified with a reporter moiety. The reporter moiety could be a fluorophore such as 5(6)-carboxyfluorescein or a quencher moiety such as 4-((4-(dimethylamino)phenyl)azo)benzoic acid (dabcyl). Condensation of the lysine derivative with the solid support can be accomplished using standard condensation (peptide) chemistry. The α-amino group of the lysine derivative can then be deprotected and the nucleobase sequence assembly initiated by condensation of the first PNA synthon with the α-amino group of the lysine amino acid. As discussed above, a spacer moiety can optionally be inserted between the lysine amino acid and the first PNA synthon by condensing a suitable spacer (e.g. Fmoc-8-amino-3,6-dioxaoctanoic acid) with the lysine amino acid prior to condensation of the first PNA synthon.

Alternatively, a functional group moiety on the assembled, or partially assembled, polymer can be introduced while the oligomer is still support bound. The functional group moiety can then be available for any purpose, including being used to either attached the oligomer to a support or otherwise be reacted with a reporter moiety, including being reacted post-ligation (by post-ligation we mean at a point after the oligomer has been fully formed by performing of one or more condensation/ligation reactions). This method, however, requires that an appropriately protected functional group moiety be incorporated into the oligomer during assembly so that after assembly is completed, a reactive functional group can be generated. Accordingly, the protected functional group can be attached to any position within the oligomer or block, including, at the oligomer termini and/or at a position internal to the oligomer.

For example, the ε-amino group of a lysine could be protected with a 4-methyl-triphenylmethyl (Mtt), a 4-methoxy-triphenylmethyl (MMT) or a 4,4′-dimethoxytriphenylmethyl (DMT) protecting group. The Mtt, MMT or DMT groups can be removed from the oligomer (assembled using commercially available Fmoc PNA monomers and polystyrene support having a PAL linker (Applied Biosystems) by treatment of the synthesis resin under mildly acidic conditions. Consequently, a donor moiety or acceptor moiety can then be condensed with the ε-amino group of the lysine amino acid while the polymer is still support bound. After complete assembly and labeling, the polymer can then cleaved from the support, deprotected and purified using well-known methodologies.

By still another method, the reporter moiety can be attached to the oligomer or oligomer block after it is fully assembled and cleaved from the support. This method is preferable where the label is incompatible with the cleavage, deprotection or purification regimes commonly used to manufacture the oligomer. By this method, the PNA oligomer can be labeled in solution by the reaction of a functional group on the polymer and a functional group on the label. Those of ordinary skill in the art will recognize that the composition of the coupling solution will depend on the nature of oligomer and label (e.g. a donor or acceptor moiety). The solution can comprise organic solvent, water or any combination thereof. Generally, the organic solvent will be a polar non-nucleophilic solvent. Non limiting examples of suitable organic solvents include acetonitrile (ACN), tetrahydrofuran, dioxane, methyl sulfoxide, N,N′-dimethylformamide (DMF) and 1-methyl pyrrolidone (NMP).

The functional group on the polymer to be labeled can be a nucleophile (e.g. an amino group) and the functional group on the label can be an electrophile (e.g. a carboxylic acid or activated carboxylic acid). It is however contemplated that this can be inverted such that the functional group on the polymer can be an electrophile (e.g. a carboxylic acid or activated carboxylic acid) and the functional group on the label can be a nucleophile (e.g. an amino acid group). Non-limiting examples of activated carboxylic acid functional groups include N-hydroxysuccinimidyl esters. In aqueous solutions, the carboxylic acid group of either of the PNA or label (depending on the nature of the components chosen) can be activated with a water soluble carbodiimide. The reagent, 1-ethyl-3-(3-dimethylamino-propyl)carbodiimide hydrochloride (EDC), is a commercially available reagent sold specifically for aqueous amide forming condensation reactions. Such condensation reactions can also be improved when 1-Hydroxy-7-azabenzotriazole (HOAt) or 1-hydrozybenzotriazole (HOBt) is mixed with the EDC.

The pH of aqueous solutions can be modulated with a buffer during the condensation reaction. For example, the pH during the condensation can be in the range of 4-10 depending on the nature of the reactive groups. Generally, the basicity of non-aqueous reactions will be modulated by the addition of non-nucleophilic organic bases. Non-limiting examples of suitable bases include N-methylmorpholine, triethylamine and N,N-diisopropylethylamine. Alternatively, the pH can be modulated using biological buffers such as (N-[2-hydroxyethyl]piperazine-N′-[2-ethanesulfonic acid) (HEPES) or 4-morpholineethane-sulfonic acid (MES) or inorganic buffers such as sodium bicarbonate.

Nucleic Acid Synthesis and Labeling

Nucleic acid oligomer (oligonucleotide and oligoribonucleotide) synthesis has become routine. For a detailed description of nucleic acid synthesis please see Gait, M. J., Oligonucleotide Synthesis: a Practical Approach. IRL Press, Oxford England. Preferably, nucleic acid oligomers are synthesized on supports in what is known as solid phase synthesis. Alternatively, they are synthesized in solution. Those of ordinary skill in the art will recognize that both labeled, unlabeled and/or modified oligonucleotides (DNA, RNA and synthetic analogues thereof) are readily available. They can be synthesized using commercially available instrumentation and reagents or they can be purchased from commercial vendors of custom manufactured oligonucleotides. Patents which discuss various compositions, supports and methodologies for the synthesis and labeling of nucleic acids include: U.S. Pat. Nos. 5,476,925, 5,453,496, 5,446,137, 5,419,966, 5,391,723, 5,391,667, 5,380,833, 5,348,868, 5,281,701, 5,278,302, 5,262,530, 5,243,038, 5,218,103, 5,204,456, 5,204,455, 5,198, 527, 5,175,209, 5,164,491, 5,112,962, 5,071,974, 5,047,524, 4,980,460, 4,923,901, 4,786,724, 4,725,677, 4,659,774, 4,500,707, 4,458,066, and 4,415,732 which are herein incorporated by reference.

PNA Chimera Synthesis and Labeling/Modification:

PNA chimeras are a combination of a nucleic acid and peptide nucleic acid subunits. Hence, the synthesis, labeling and modification of PNA chimeras can utilize methods known to those of skill in the art as well as those described above. A suitable reference for the synthesis, labeling and modification of PNA chimeras can be found in WIPO published patent application number WO96/40709, now issued as U.S. Pat. No. 6,063,569, herein incorporated by reference. Moreover, the methods described above for PNA synthesis and labeling often can be used for modifying the PNA portion of a PNA chimera. Additionally, well-known methods for the synthesis and labeling of nucleic acids can often be used for modifying the nucleic acid portion of a PNA chimera.

Labels:

The labels attached to the component polymers of the complexes used as primers in method embodiments of this invention can comprise a set (hereinafter “beacon set(s)”) of energy transfer moieties comprising at least one donor and at least one acceptor moiety. Typically, the beacon set will include a single donor moiety and a single acceptor moiety. Nevertheless, a beacon set can contain more than one donor moiety and/or more than one acceptor moiety. For example, a set could comprise three moieties. Moiety one can be a donor fluorophore which, when exited and located in close proximity to moiety two, can then transfer energy to moiety two of the beacon set. Thereafter, moiety two, which when excited and located in close proximity to moiety three, can transfer energy to moiety three of the beacon set. Consequently, energy is transferred between all three moieties of this beacon set. In this set, moiety two is both an acceptor of energy from moiety one and a donor of energy to moiety three. Such transfers of energy between two or more moieties of a beacon set are contemplated by the practice of the embodiments of this invention.

The donor and acceptor moieties operate such that one or more acceptor moieties accepts energy transferred from the one or more donor moieties or otherwise quench signal from the donor moiety or moieties. Transfer of energy can occur through collision of the closely associated moieties of a beacon set (non-FRET; See: Yaron et al. Analytical Biochemistry, 95, 228-235 (1979) and particularly page 229, col. 1 through page 232, col. 1), through a nonradiative process such as fluorescence resonance energy transfer (FRET; See: Yaron et al. Analytical Biochemistry, 95, 228-235 (1979) and particularly page 232, col. 1 through page 234, col. 1), a combination of the foregoing or through other as yet unexplained mechanisms. Accordingly, the mechanism of energy transfer is not a limitation of the embodiments of this invention as energy transfer between the donor and acceptor molecules can occur in any way.

Donor and acceptor moieties can be fluorophore and quencher combinations. Numerous amine reactive fluorophore and quencher labeling reagents are commercially available (as for example from Molecular Probes, Eugene, Oreg.). Labeling reagents can be supplied as carboxylic acids or as the N-hydroxysuccinidyl esters of carboxylic acids. Preferred fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.) or the Alexa dye series (Molecular Probes, Eugene, Oreg.). A quencher moiety is a moiety that can quench detectable signal from a donor moiety such as a fluorophore. The quencher moiety can be another fluorophore. The quencher moiety can be an aromatic or heteroaromatic moiety that is substituted with one or mores azo or nitro groups. For example, the quencher moiety can be 4-((-4-(dimethylamino)phenyl)azo)benzoic acid (dabcyl).

Multiple Sets of Donor and Acceptor Moieties

Multiple beacon sets can be easily incorporated into the primer complexes because the individual component polymers can incorporate multiple labels wherein each of the different labels of an individual component polymer pertain to a member of a different beacon set. For example, each of one annealing polymer and one priming polymer can be labeled at both termini with either of a donor fluorophore or quencher acceptor moiety. Consequently, two beacons sets can be present in a single primer complex. Assuming that two of the same fluorophore and two of the same quencher moieties are present, at least three combinations exist for the primer complex. In one combination, both fluorophores are attached to the priming polymer and both quenchers are attached to the annealing polymer. In the second combination, both quenchers are attached to the priming polymer and both fluorophores are attached to the annealing polymer. In the third combination, one fluorophore and one quenching moiety is attached to the priming polymer and one fluorophore and one quenching moiety is attached to the annealing polymer. The only caveat is that energy transfer occurs between members of each beacon set in the assembled primer complex.

In some embodiments, the fluorophores are different. Preferably, the different fluorophores used in each beacon set are independently detectable. Consequently, multiple beacon sets containing two or more independently detectable donor and/or acceptors moieties offer even greater diversity to the useful applications of primer complexes. These primer complexes are particularly well suited for use in multiplex assays wherein detectable signal from each of the independently detectable fluorophores can be correlated with activity associated with the polymerase extension of the primer complexes.

It follows that the principles discussed above can be used to prepare numerous alternative embodiments of primer complexes that incorporate multiple component polymers and multiple sets of donor and acceptor moieties. Numerous alternative embodiments of primer complexes in combination with the multiple independently detectable moieties are contemplated as part of the embodiments of this invention.

Detection of Energy Transfer:

When a complex that is to be used as a primer (a primer complex) is formed, at least one donor moiety of one component polymer is brought sufficiently close in space to at least one acceptor moiety of a second component polymer. Since the donor and acceptor moieties of the set can be closely situated in space, transfer of energy occurs between moieties of the beacon set. When the primer complex dissociates, the donor and acceptor moieties do not interact sufficiently to cause substantial transfer of energy from the donor and acceptor moieties of the beacon set and there is a correlating change in detectable signal from the donor and/or acceptor moieties of the set. Consequently, primer complex formation/dissociation can be determined by measuring at least one physical property of at least one member of the beacon set that is detectably different when the complex is formed as compared with when the component polymers of the primer complex exist independently or dissociated.

Detectable and Independently Detectable Moieties/Multiplex Analysis:

In preferred embodiments of this invention, a multiplex hybridization assay can be performed. In a multiplex assay, numerous conditions of interest can be simultaneously examined. Multiplex analysis relies on the ability to sort sample components or the data associated therewith, during or after the assay is completed. In some embodiments of the invention, distinct independently detectable moieties can be used to label component polymers of two or more different primer complexes. The ability to differentiate between and/or quantitate each of the independently detectable moieties facilitates the method embodiments of this invention because the data that correlates with the priming activity (and the associated amplification activity) of each of the distinctly (independently) labeled primer complex can be correlated with information pertaining to the state of degradation and/or concentration of the target nucleic acid strand sought to be determined.

Because the primer complexes can be self-indicating, and can be designed to be independently detectable, the multiplex assays of the embodiments of this invention can be performed in a closed tube format to provide data for real-time or end-point analysis of a sample. By self-indicating we mean that the there is a detectable change that occurs as a result of the operation of the method such that there is no requirement for additional physical manipulation of the sample that is unrelated to the operation of the amplification process (e.g. removal of excess primer complex). By independently detectable we mean that it is possible to determine one label independently, and optionally, in the presence of another label.

Spacer/Linker Moieties:

Generally, spacers can be used to minimize the adverse effects that bulky labeling reagents might have on hybridization properties of non-nucleic acid probes. Linkers can induce flexibility and randomness into a nucleobase polymer or otherwise link two or more nucleobase sequences of a nucleobase polymer. Preferred spacer/linker moieties for non-nucleic acid polymers (e.g. PNA) consist of one or more aminoalkyl carboxylic acids (e.g. aminocaproic acid) the side chain of an amino acid (e.g. the side chain of lysine or ornithine) natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane). Spacer/linker moieties can also incidentally or intentionally be constructed to improve the water solubility of the probe (For example see: Gildea et al., Tett. Lett. 39: 7255-7258 (1998)). Preferably, a spacer/linker moiety comprises one or more linked compounds having the formula: —Y—(Oₘ—(CW₂)ₙ)ₙ-Z-. The group Y has the formula: a single bond, —(CW₂)ₚ—, —C(O)(CW₂)ₚ—, —C(S)(CW₂)ₚ— and —S(O₂)(CW₂)ₚ. The group Z has the formula NH, NR², S or O. Each W is independently H, R², —OR², F, Cl, Br or I; wherein, each R² is independently selected from the group consisting of: —CX₃, —CX₂CX₃, —CX₂CX₂CX₃, —CX₂CX(CX₃)₂, and —C(CX₃)₃. Each X is independently H, F, Cl, Br or I. Each m is independently 0 or 1. Each n, o and p are independently integers from 0 to 10.

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a primer hybridization is often found by the well known technique of fixing several of the aforementioned stringency factors and then determining the effect of varying a single stringency factor. The same stringency factors can be modulated to thereby control the stringency of hybridization of non-nucleic acid polymers such as PNA, except that the hybridization of a non-nucleic acid polymer (e.g. PNA) can be fairly independent of ionic strength. Optimal stringency for an assay can be experimentally determined by examination of each stringency factor until the desired degree of discrimination is achieved.

The aforementioned stringency factors can also apply to the formation and/or dissociation of the primer complexes since they comprise two or more hybridized component nucleobase polymers. Consequently, control of stringency factors can allow one to preferentially modulate the stability of the primer complex with respect to the primer site of the target nucleic acid molecule in a controlled fashion to thereby achieve additional advantages and benefits.

Suitable Priming Conditions:

It is well known that polymerase extension reactions can be primer mediated. Those of ordinary skill in the art will appreciate how to achieve suitable priming conditions for a polymerase enzyme.

Generally however, a primer hybridizes to a template and then a polymerase extends the primer, consuming nucleotide triphosphates by operation of the polymerase extension. Thus, suitable priming conditions include suitable hybridization conditions since the primer must hybridize to the priming site. Additionally, the temperature, salt concentration (ionic strength), magnesium concentration and concentration of nucleotide triphosphates can be adjusted based upon the polymerase chosen for the primer extension reaction. Often the supplier of the polymerase can provide useful information on optimal primer conditions or even provide reagents and/or kits tailored for use with the polymerase enzyme. When information is not provided, it is possible for the ordinary practitioner to determine suitable priming conditions essentially as described above for the determination on suitable hybridization conditions. For example, one factor is varied whilst others are held constant until suitable priming conditions are determined.

Amplification Reactions:

The polymerase extension reactions can be used in method embodiments of this invention to both generate amplified molecules as well as to cause dissociation of the primer complexes. The polymerase extension reactions perform as part of a nucleic acid amplification methodology. Non-limiting examples of suitable nucleic acid amplification reactions include: Polymerase Chain Reaction (PCR), Ligase Chain Reaction (LCR), Strand Displacement Amplification (SDA), Transcription-Mediated Amplification (TMA), Q-beta replicase amplification (Q-beta) and Rolling Circle Amplification (RCA).

PCR Clamping:

PCR Clamping is a method (not necessarily limited to PCR applications; see U.S. Pat. No. 5,891,625 at col. 2, lines 5-37) wherein the amplification of one or more possible nucleic acid sequences in the sample can be disabled using one or more PNA, or other non-nucleic acid polymers. The process is more fully described in U.S. Pat. Nos. 5,891,625 and 5,972,610, herein incorporated by reference. Briefly, the specific disabling of amplification of certain nucleic acid molecules can be used for example, often by way of an internal control, for single point mutation analysis and/or for improving the reliability of the result of an amplification process. Basically amplification is inhibited because the PNA, or other non-nucleic acid polymer, hybridizes to the target nucleic acid to be amplified at a position that either inhibits the primer from hybridizing to the primer site or inhibits the proper extension of the polymerase. By either method, amplification of a segment of a specific target nucleic acid molecule can be inhibited.

4. Description of Various Embodiments of the Invention:

Primer Complexes:

Primer complexes are hybrids of at least two component polymers. At least two of the component polymers of the primer complex can comprise at least one moiety from a set of donor and acceptor moieties (a beacon set as previously described), though a primer complex can comprise more than one beacon set. Component polymers can be designed to form the primer complex by the interaction of interacting groups. The primer complex can comprise one or more linkers and/or one or more spacer moieties as can be useful to construct a primer complex suitable for a particular assay.

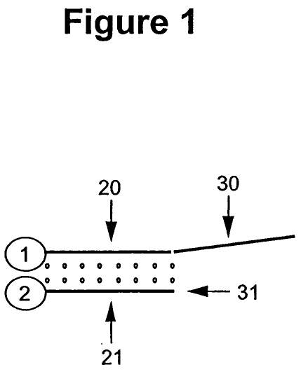

Each primer complex comprises at least one priming polymer and at least one annealing polymer. The priming polymer contains a sequence of subunits suitable for hybridizing to a priming site under suitable priming conditions. The annealing polymers primary utility will be to form the primer complex, though the annealing polymer can, in some embodiments, also be an information determining polymer. By “information determining polymer” we mean that the polymer sequence specifically binds to a target sequence in the sample and that binding event is determined. Interacting groups cause the priming polymer to anneal to the annealing polymer(s) to thereby form and stabilize the primer complex. Any combination of priming and annealing polymers can be constructed with the appropriate interacting groups (See for example the illustrations, and accompanying description, of FIGS. 1 and 1 of U.S. Pat. No. 6,361,942, incorporated herein by reference). Moreover, the priming and annealing polymers can be PNA, DNA, RNA or chimeric oligomers. In some embodiments, at least one of the component polymers can be a non-nucleic acid polymer. The non-nucleic acid polymer can be a peptide nucleic acid (PNA).

With reference to FIG. 1 herein, a simple example of a primer complex comprising a single donor and single acceptor moiety is illustrated. The primer complex comprises a single set of donor and acceptor moieties (1 and 2), each of which is linked to a different component polymer. The donor and acceptor moieties 1 and 2 represent the beacon set. As illustrated, the interacting groups (20 interacting with 21) comprise nucleobase sequences of the priming polymer (30) and the annealing polymer (31). The primer complex is formed and stabilized by the interaction of the interacting groups. As illustrated, the annealing polymer (31) comprises only the interacting groups (21) necessary to form a stable complex with the priming polymer (30) and therefore the annealing polymer (31) is shorter than the priming polymer (30). Because the priming polymer (30) can form a stable hybrid with the priming site, the priming polymer (30) can typically be designed to be longer than the annealing polymer (31).

For example, the component annealing polymer of each primer complex illustrated in FIG. 1 can be a C-terminal dabcyl labeled PNA oligomer that is hybridized to a 5′-fluorophore labeled nucleic acid priming polymer. Since it is possible to invert the labels with respect to the polymers, in some embodiments, each primer complex illustrated in FIG. 1 can be a C-terminal fluorophore labeled PNA oligomer that is hybridized to a 5′-quencher labeled nucleic acid priming polymer.

As will be discussed in more detail below, the primer complexes of the embodiments of this invention can be designed to dissociate as an indirect or direct consequence of the hybridization of the priming polymer to a priming site. We say that the dissociation is direct when the primer complex dissociates essentially upon hybridization of the priming polymer to the priming site. We say that the primer complex dissociates as an indirect consequence of hybridization when a secondary triggering event causes dissociation but mere hybridization to the priming site does not substantially result in dissociation.

Because the component polymers of a primer complex dissociate (either directly or indirectly from hybridization to a primer site), the attached donor and acceptor moieties, which are independently attached to different polymers, can become substantially separated in space. As a consequence of this separation in space, the efficiency of energy transfer can be changed or be virtually eliminated upon dissociation. Because the primer complexes are self indicating, a resulting change in the signal from the donor, the acceptor or both the donor and acceptor can be used to measure the operation of the amplification as it proceeds in real-time or at the end-point. Direct and indirect dissociation of such complexes are described in copending and commonly owned U.S. Pat. Nos. 6,361,942 and 6,607,889, incorporated herein by reference.

By way of example, a typical assay performed in accordance with the embodiments of this invention might involve determining the increase in fluorescence during (i.e. real-time), or at the end-point of the assay, wherein an increase in fluorescence correlates with activity of the amplification reaction. Quantitation of the target nucleic acid molecule in the sample, or portion thereof, that is amplified can be determined by comparison with a standard curve generated using a standardized procedure and known quantities of the target nucleic acid molecule in a representative sample.

(i) Priming Polymers:

Priming polymers comprise at least one priming segment. The priming segment of the primer complex is a sequence specific recognition portion of the construct. Therefore, the priming segment is a nucleobase sequence containing subunits designed to hybridize to a specific priming site, under suitable hybridization conditions, and operate as a primer under suitable priming conditions. The entire priming polymer can comprise the priming segment wherein the interacting groups are integral to the priming segment. Alternatively, the priming polymer can contain both a priming segment and also one or more interacting groups, wherein the interacting groups do not hybridize to or otherwise interact with the priming site of a target nucleic acid molecule.

With due consideration of the requirements of a priming polymer, the length of the priming segment can be chosen such that a stable complex is formed between the priming segment and the priming site under suitable priming conditions. The priming segment can have a length of about 5 to about 50 nucleobase containing subunits. The priming segment can have a length of about 7 to about 25 nucleobase containing subunits in length. The priming segment can have a length of about 12 to about 20 nucleobase containing subunits in length.

The priming segment of a primer complex can have a nucleobase sequence that is complementary to the target sequence. However, a substantially complementary priming segment might be used since it has been demonstrated that greater sequence discrimination can be obtained when utilizing probes wherein there exists a single point mutation (base mismatch) between the probe and the target sequence (See: Guo et al., Nature Biotechnology 15: 331-335 (1997)).

The priming polymer can comprise at least one member of the beacon set. However, the priming polymer need not comprise a member of the beacon set where the primer complex comprises at least two annealing component polymers. In this case, each of the annealing polymers can comprise one of the donor or acceptor moieties and the priming polymer can be unlabeled.

The priming polymer can be a nucleic acid because polymerases, transcriptases and ligases are known to operate on nucleic acid complexes. However, modified PNAs or chimeric PNAs are also known to be substrates for certain enzymes provided they are modified to comprise the necessary functional group or groups (e.g. a 3′-hydroxyl group; See: Lutz et. al., J. Am. Chem. Soc., 119: 3171-3178 (1997)). Thus, the priming polymer can be a modified non-nucleic acid polymer, such as a PNA chimera. In some embodiments, the primer complex is constructed from one nucleic acid priming polymer and one non-nucleic acid annealing polymer. The non-nucleic acid annealing polymer can be a PNA oligomer.

(ii) Annealing Polymers:

One or more annealing polymers anneal to a priming polymer to thereby form a primer complex. Thus, an annealing polymer can, in some embodiments, merely be used to form the primer complex. Alternatively, the annealing polymer can itself be an information-determining polymer. At a minimum, an annealing polymer comprises interacting groups necessary for the formation of the primer complex when annealed to the priming polymer though at least one annealing polymer of a primer complex must have at least one linked member of the beacon set.

In some embodiments, the same annealing polymer can be used to form two or more primer complexes. For example, the annealing polymer can be a C-terminal dabcyl labeled PNA oligomer that is capable of hybridizing to the interacting groups of two or more different 5′-fluorsecently labeled nucleic acid oligomers to thereby form the two or more different primer complexes. (A C-terminal dabcyl labeled PNA oligomer designed to hybridize to a complementary, or partially complementary, fluorescently labeled oligomer (PNA, nucleic acid or chimera) and thereby quench the fluorescence of the fluorophore is sometimes referred to as Q-PNA) For example, the two or more different primer complexes can be used in the same assay such that only one (the common) annealing polymer is present in the assay.

(iii) Interacting Groups:

Interacting groups are the moieties of component polymer(s) that form and stabilize the primer complex. The interacting groups can comprise the entirety of the priming and/or annealing polymer(s). Alternatively, the interacting groups can comprise only a subset of the subunits of one or both of the priming and/or annealing polymer(s).

In some embodiments, interacting groups can be hydrophobic moieties such as the fluorophores and quenchers or otherwise comprise both fluorophore(s) and quencher(s) in concert with other lipophilic moieties. In some embodiments, the interacting groups can be ionized groups that form salt pairs. One example of such a salt pair would comprise the interaction of positively charged c-amino group(s) of one or more lysine moieties paired with negatively charged side chain carboxylic acid group(s) of one or more aspartic acid or glutamic acid moieties. In some other embodiments, the interacting groups can comprise hydrogen bonding moieties. For example, the interacting groups can be complementary nucleobases of all, or a portion, of the nucleobase sequence of the component polymers. Formation and stability of the primer complex will inevitably be affected by all of the hydrophobic, ionic and/or hydrogen bonding properties of the all moieties of the component polymers.

In some embodiments, interacting groups can be located at only one terminus of each of the priming and annealing polymers (e.g. FIG. 1). In some embodiments, interacting groups can be linked to both termini of each of the component polymers. In some embodiments, interacting groups can be internal (e.g. centered within one or more component polymers) to one or more of the component polymers. In some embodiments, the interacting groups are located at one or more termini and internal to one or more of the component polymers.

(iv) Base Pairing Motifs:

When the interacting groups comprise nucleobases, any base pairing motif that can form a stable complex of at least two component polymers of a primer complex can be used to form the primer complex. Non-limiting examples of suitable base pairing motifs include duplexes, triplexes as well as other multimers and higher order structures that nucleic acids, nucleic acid analogs, nucleic acid mimics and/or chimeras can adopt to form a complex.

(v) Equilibrium Factors:

Primer complexes can be hybrids that are formed in solution by mixing the component polymers under conditions favorable for complex formation (e.g. hybridization). Because the quantity of each component polymer added to form the primer complex can be calculated and controlled, the extent of complex formation/dissociation can be manipulated or controlled by adjusting the quantity or concentration of the two or more component polymers present in the assay to thereby reduce, or otherwise adjust for, background.

For example, if a priming polymer, containing a single fluorophore, and an annealing polymer, containing a single quenching moiety, are 95 % associated when in a ratio of 1:1 (at a given concentration), but, are 99.9% associated when mixed in a ratio of 1:5 (at a given concentration), the sample containing the 1:1 ratio can have a substantial background fluorescence attributable to the 5% fluorescently labeled priming polymer which is free in solution. However, the sample containing a 1:5 ratio can have substantially less inherent fluorescence because essentially the entire fluorophore-containing polymer is completely complexed to the quencher moiety containing annealing polymer. Consequently, the background fluorescence of any solution containing primer complexes can be adjusted by altering the relative ratios and concentrations of component polymers to thereby adjust the extent of hybrid formation. Based on the foregoing it will be apparent to the ordinary practitioner that the ratio can change during the operation of an assay and this fact should be considered in the assay design.

It follows that the background fluorescence of the assays utilizing a primer complex can also be favorably modulated by adjusting the concentration of component polymers with regard to the amount of target nucleic acid molecule in a sample. Extending the example analysis above, we can assume that a solution used to analyze a sample for target sequence comprises a 1:1 ratio of priming and annealing polymers to thereby produce a solution containing 95% of the primer complex. If we assume that 80% of the priming polymer becomes annealed preferentially to a target nucleic acid molecule of the sample thereby resulting in dissociation of the primer complex, the relative ratio of priming and annealing polymers which are still free in solution to form the primer complex is then 1:5 (all fluorophore containing probe is now 99.9% associated to a quencher containing probe). This shift in equilibrium can result in a significant reduction in the background fluorescence of the system. It thereby follows that the judicious choice of the concentration or amount of each of the component polymers added to an assay can enhance the practice of the method embodiments of this invention. As stated above, the likelihood that the ratio of component polymers can change during the operation of the assay is a factor to be considered. Furthermore, because non-nucleic acid polymers, such as unmodified PNA oligomers, can bind more strongly to nucleic acid than does a nucleic acid polymer, use of one or more PNA polymers as the component polymers of the primer complex can be advantageous where less excess of a polymer is needed to drive the equilibrium toward essentially complete formation of the primer complex.

(vi) Formation and Stability of Primer Complexes

Component polymers can be constructed so that multiple types of interacting groups are present and contribute to the stability/lability of the assembled primer complex (See discussion in section entitled: “Interacting Groups”). The composition of the component polymers can be judiciously chosen so that the primer complex is stable under predefined conditions and dissociates under predefined conditions. Generally, the thermodynamic parameters for the stability and lability of a primer complex can be determined by examination of melting point since those of skill in the art will recognize that Tm analysis can be used to determine the ΔH, ΔS and ΔG values for formation and dissociation of a hybrid of two or more polymers. The practice is so common that the software that accompanies automated instrumentation is typically equipped to derive the ΔH, ΔS and ΔG values after the Tm analysis is performed.

Choosing a primer complex of proper stability can be an important consideration in the design of assays for real-time or end-point analysis of a sample. It is important to note however, that real-time analysis must be made under conditions where the primer complex is free to form so that detectable signal changes are properly attributable to the amplification of target nucleic acid molecule and not to conditions that induce target independent dissociation. Using the disclosure provided herein as well as the disclosure in U.S. Pat. No. 6,361,942 (incorporated herein by reference), those of skill in the art will require no more than routine experimentation to design component polymers for forming primer complexes of suitable stability/lability for particular applications.

Though the composition of the interacting groups can be a primary factor used to influence the stability/lability of the complex, the length and composition of the priming polymer can influence complex stability. Specifically, since hybridization is a cooperative event, the hybridization of the priming segment to a primer site can sterically or functionally influence the interactions of interacting groups linked thereto. Consequently, these types of secondary effects should also be considered when choosing the nature of the component polymers of the primer complex.

The primer complex can be designed to remain intact after the priming segment hybridizes to the priming site. In this configuration, there are typically no interacting groups of the priming polymer that also contribute to the formation of the hybrid between the priming segment and the primer site. For this reason, the thermodynamic stability of the hybrid formed from the priming segment and primer site is typically unrelated to the thermodynamic stability of the interacting groups which form the primer complex. Consequently, the primer complex of this configuration can be constructed so that a secondary “triggering” event disrupts the interactions of the interacting groups to thereby cause dissociation of the primer complex. The secondary triggering event can be the operation of the amplification process.

The primer complex can be designed to dissociate upon hybridization of the priming segment to the priming site. In this configuration, there are typically one or more interacting groups of the priming polymer that also contribute to the formation of the hybrid with the primer site. By design, the complex formed between the priming segment and the primer site is thermodynamically favored as compared with the stability of the primer complex. Since the initial interactions by primer segment with the primer site can result in cooperative binding of all or most of the complementary sequence of the priming polymer, the interactions can necessarily cause the release of the annealing polymer or polymers and dissociation of the primer complex. By this method, dissociation of the primer complex occurs directly as a result of hybridization of the priming polymer of the primer complex.

(vii) Secondary “Triggering” Events:

Secondary “triggering” events can be operations or events that indirectly can cause the primer complex to dissociate after it has annealed with the priming site of a target nucleic acid molecule. In embodiments of the present invention, an appropriate secondary “triggering” event can be a nucleic acid amplification reaction wherein the complex formed between the priming segment and the priming site of the target nucleic acid molecule is the substrate for a polymerase. For example, the polymerase extension reaction, as a secondary triggering event, can operate in a PCR amplification reaction as illustrated in FIGS. 2-5.

With reference to FIG. 2A, a double stranded plasmid template (6) is illustrated. As illustrated, both of the forward (6l) or reverse (62) primers are primer complexes although only one of the two primers need be a primer complex. However, where both PCR primers are primer complexes, a common annealing polymer can be used. (See: U.S. Pat. Nos. 6,361,942 or 6,607,889) for a discussion of the use of common annealing polymers) Again with reference to FIG. 2, an expansion of the polymerase reaction initiated by the forward primer (61) is illustrated in FIG. 2B. As illustrated, a single strand of the plasmid (63) comprising the priming site (64) is shown. The polymerase (65) thereby generates the complementary strand of the plasmid. As illustrated, the priming segment (66) of the priming polymer (68) hybridizes to the priming site (64). The annealing polymer (69) hybridizes to the interacting groups (67) of the priming polymer (68) to thereby form the primer complex. As illustrated, the primer complex does not dissociate upon hybridization to the priming site.

First round products of PCR amplification are illustrated in FIG. 3. As illustrated, both the forward (70) and reverse primers (71) are primer complexes. Complementary strands of the plasmid are illustrated as 73 and 74. The polymerase (65) can copy the plasmid until it detaches. As illustrated, the primer complexes are not yet dissociated. Therefore each fluorophore F6 and F7 is quenched by the quencher moieties Q2 and Q3, respectively.

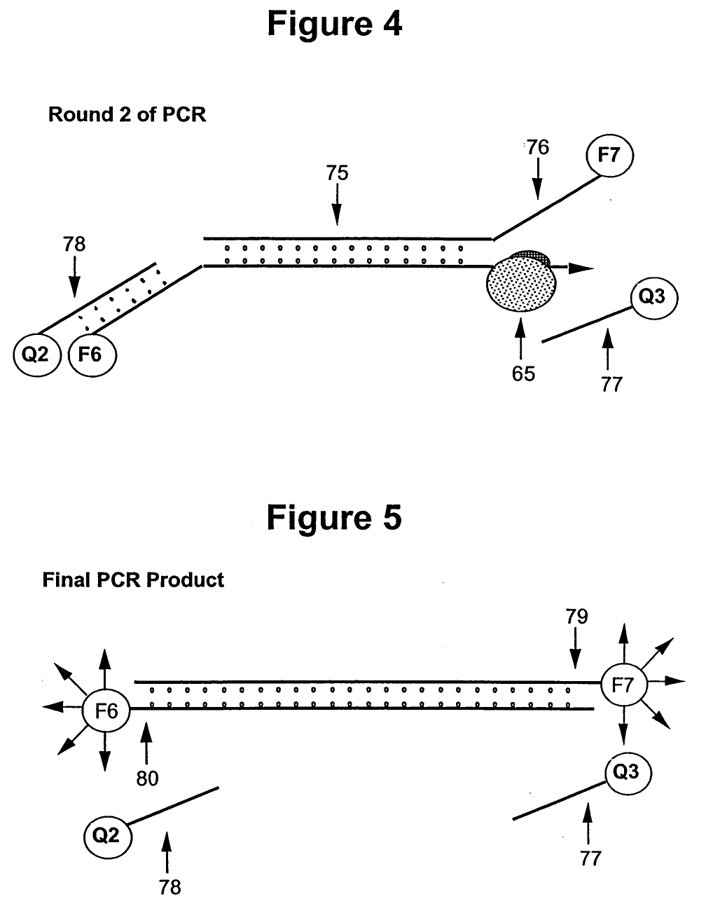

The second round of PCR is illustrated in FIG. 4. With reference to FIG. 4, the polymerase enzyme copies back on the round one polymerization product (75). As it reaches the end of the template strand, the polymerase (65) can push the annealing polymer (77) off the template thereby dissociating the primer complex and releasing the annealing polymer into the solution. Consequently, it is the operation of the PCR process that causes indirect dissociation of the primer complex. Thus, the PCR process in the secondary “triggering” event that causes indirect dissociation of the primer complex.

With reference to FIG. 4, it becomes apparent that if the primer polymer is a nucleic acid and the interacting groups (76) comprise nucleotides, then the polymerase can copy past the hybridization site of the probing segment of the primer complex to thereby extend the amplicon past the original priming site(s) of the template. This process thereby generates an amplicon that is longer than the distance between the termini of the priming sites on the target nucleic acid molecule.

With reference to FIG. 5, the final PCR amplicon is illustrated. As illustrated, the fluorescence of each fluorophore (F6 & F7) at each terminus of the amplicon (79 and 80) can be detected since the primer complexes have been dissociated thereby releasing into solution the annealing polymers (77 and 78) comprising the quencher moieties (Q2 & Q3). As previously discussed, the fluorophores and quencher moieties can be the same or different. Moreover, a single assay can be multiplexed to thereby detect, identify and/or quantitate two or more unique target sequences. When multiplexing, it is possible to use common annealing polymers comprising a quencher moiety and nucleic acid primers for two or more target nucleic acid molecules of interest wherein one or both of the primers of each set are labeled with independently detectable moieties such that production of the amplicons for each unique target molecule are independently detectable.

From the above-described example, it becomes clear that for some embodiments of indirect dissociation of the primer complex to occur, the amplification process can be designed so that a polymerase can copy back on the nucleic acid strand that is created by polymerase extension of the primer complex. In this way, copying of, all or part of, the priming polymer can release the annealing polymer and thereby result in dissociation of the primer complex.

Immobilization of a Primer Complex to a Surface:

One or more of the component polymers which comprise a primer complex can optionally be immobilized to a surface for the purpose of forming a support bound primer complex. For example the component priming polymer can be immobilized or tethered to the surface. Alternatively, one annealing polymer can be immobilized or tethered to the surface.

The component polymer can be immobilized to the surface using, for example, the process of UV-crosslinking. Alternatively, the component polymer can be synthesized on the surface in a manner suitable for deprotection but not cleavage from the synthesis support (See: Weiler, J. et al., “Hybridization based DNA screening on peptide nucleic acid (PNA) oligomer arrays”, Nucl. Acids Res., 25:, 2792-2799 (1997)). In still another embodiment, one or more component polymer can be covalently linked to a surface by the reaction of a suitable functional group on the polymer with a functional group of the surface (See: Lester, A. et al, PNA array technology. Presented at Biochip Technologies Conference in Annapolis (October, 1997)).

Methods for the attachment of polymers to surfaces generally involve the reaction of a nucleophilic group, (e.g. an amine or thiol) of the polymer to be immobilized, with an electrophilic group on the support to be modified. Alternatively, the nucleophile can be present on the support and the electrophile (e.g. activated carboxylic acid) present on the polymer. Because native PNA and other non-nucleic acid probes can possess an amino terminus, they may not necessarily require modification to be immobilized to a surface (See: Lester et al., Poster entitled “PNA Array Technology”). Conversely, nucleic acid probes can generally be prepared in modified form (e.g. prepared as amine or thiol modified polynucleotides) using commercially available reagents and/or supports, if they are to be immobilized to a support.

Conditions suitable for the immobilization of a nucleic acid or PNA to a surface can generally be similar to those conditions suitable for the labeling of the polymer. The immobilization reaction is essentially the equivalent of labeling whereby the label is substituted with the surface to which the polymer is to be covalently immobilized. Numerous types of surfaces derivatized with amino groups, carboxylic acid groups, isocyantes, isothiocyanates and malimide groups are commercially available. Methods to label nucleic acid and non-nucleic acid polymers have been previously described herein.

Once a single component polymer is immobilized to a surface, the primer complex can be formed simply by contacting the surface with a solution containing the other component polymer or polymers under conditions suitable for the complex to assemble (e.g. suitable hybridization conditions). When immobilized to a surface, the self-indicating primer complex can exhibit little or no detectable signal until the component polymers are dissociated. The ability to retain one of the component polymers on a surface while releasing other component polymers into solution can facilitate detectable changes in signal of the donor and acceptor moieties.

Methods:

The state of degradation and/or total concentration of nucleic acid of a sample is of interest in various forensic applications as well as applications, including without limitation, for the purpose dating samples. Some embodiments of the present invention provide methods for determining or estimating the state of degradation of nucleic acid in a sample or provide methods for determining or estimating the total nucleic acid in an unknown sample. The nucleic acid to be determined or estimated can be species specific (e.g. from human, mouse, pig, etc.)

Thus, in some embodiments, this invention pertains to a method for determining or estimating the state of degradation of nucleic acid in a sample, or one or more portions thereof. According to the method, a sample, or one or more portions thereof, comprising degraded nucleic acid is contacted with at least two amplification primer sets wherein at least one of the primers of each set is an independently detectable primer complex. The independently detectable primer complex comprises at least one component priming polymer wherein the priming polymer comprises: 1) a priming segment that is complementary, or substantially complementary, to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site. The independently detectable primer complex further comprises at least one component-annealing polymer that, at a minimum, comprises one or more interacting groups wherein the interacting groups of the component polymers form and stabilize the primer complex. The independently detectable primer complex further comprises at least one independently detectable set of donor and acceptor moieties wherein to each of at least two of the component polymers of each independently detectable primer complex is linked at least one moiety from each set such that formation of the primer complex facilitates transfer of energy between donor and acceptor moieties of the set.

According to some embodiments of the method, the sample, or one or more portions thereof, can be contacted with at least the other reagents required to perform a nucleic acid amplification reaction. The nucleic acid amplification reaction can be performed wherein the two or more primer complexes can be used as primers and wherein the nucleobase sequence of the primers, or component priming polymers of the amplification primer sets, can be selected to amplify the same or different target nucleic acid molecules of the sample such that one amplified molecule is shorter than at least one other amplified molecule. Changes in detectable signal attributable to the changes in the transfer of energy between the donor and/or acceptor moieties of each independently detectable primer complex can be determined. These results, for each different independently detectable primer complex, can be compared with one or more control samples and/or with a standard curve to thereby determine or estimate the state of degradation of the nucleic acid of the sample.

Because independently detectable primer complexes can be used, these assays can be performed in multiplex mode in the same tube but individual amplification reactions, each with a single primer set, can also be performed in separate containers, vessels or tubes. In some embodiments, a portion of the sample is contacted with one of the at least two amplification primer sets in a separate container, vessel or tube. In some embodiments, each of the at least two amplification primer sets is contacted with a portion of the sample in a separate container, vessel or tube. In some embodiments, the assay is performed in a single tube on the sample, or a portion thereof.

In some embodiments, it is desirable to repeat the same assay more than one time so that average of the various assays can be used as a result. Thus, in some embodiments, the above described method can be performed on each of two or more portions of the same sample wherein the average or mean of the result obtained for each individual measurement is used to determine or estimate the state of degradation of the nucleic acid of the entire sample.

In still some other embodiments, this invention pertains to a method for determining or estimating the state of degradation of nucleic acid in each of at least two portions of a sample. According to the method, each of two or more portions of a sample comprising degraded nucleic acid is contacted with a different amplification primer set wherein at least one of the primers of each set is a primer complex. Each primer complex comprises at least one component priming polymer wherein the priming polymer comprises: 1) a priming segment that is complementary, or substantially complementary, to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site. Each primer complex further comprises at least one component-annealing polymer that, at a minimum, comprises one or more interacting groups wherein the interacting groups of the component polymers form and stabilize the primer complex. Each primer complex further comprises at least one set of donor and acceptor moieties wherein to each of at least two of the component polymers of each primer complex is linked at least one moiety from each set such that formation of the primer complex facilitates transfer of energy between donor and acceptor moieties of the set.

According to some embodiments, each sample portion can be contacted with at least the other reagents required to perform a nucleic acid amplification reaction. The nucleic acid amplification reaction can be performed on each sample portion wherein the two or more primer complexes can be used as primers and wherein, in the at least two sample portions, the nucleobase sequence of the primers, or component priming polymers of the amplification primer sets, can be selected to amplify the same or different target nucleic acid molecules of each sample portion such that, in one of the at least two sample portions, one amplified molecule is shorter than at least one other amplified molecule in another sample portion. Changes in detectable signal attributable to the changes in the transfer of energy between the donor and/or acceptor moieties of each primer complex can be determined. These results, for each primer complex, can be compared with one or more control samples and/or with a standard curve to thereby determine or estimate the state of degradation of the nucleic acid of the sample.

If the above-described method utilizes independently detectable primer sets, the result of more than one amplification reaction can be determined in a single tube. Accordingly, in some embodiments, a portion of the sample is contacted with one of the at least two amplification primer sets in a separate container, vessel or tube. In some embodiments, each of the at least two amplification primer sets is contacted with a portion of the sample in a separate container, vessel or tube.

In some embodiments, the above described method can be performed on repeats of each of two or more portions of the same sample wherein the average or mean of the result obtained for each individual measurement is used to determine or estimate the state of degradation of the nucleic acid of the entire sample.

In still some other embodiments, this invention pertains to a method for determining or estimating the concentration of amplifiable nucleic acid in a sample, or one or more portions thereof. According to the method, a sample, or one or more portions thereof, comprising nucleic acid is contacted with at least two amplification primer sets wherein at least one of the primers of each set is an independently detectable primer complex. The independently detectable primer complex comprises at least one component priming polymer wherein the priming polymer comprises: 1) a priming segment that is complementary or substantially complementary to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site. The independently detectable primer complex further comprises at least one component-annealing polymer that, at a minimum, comprises one or more interacting groups wherein the interacting groups of the component polymers form and stabilize the primer complex. The independently detectable primer complex still further comprises at least one independently detectable set of donor and acceptor moieties wherein to each of at least two of the component polymers of each independently detectable primer complex is linked at least one moiety from each set such that formation of the primer complex facilitates transfer of energy between donor and acceptor moieties of the set.

According to some embodiments, the sample, or one or more portions thereof, can be contacted with at least the other reagents required to perform a nucleic acid amplification reaction. The nucleic acid amplification reaction can be performed on the sample, or one or more portions thereof, wherein the two or more primer complexes can be used as primers and wherein the nucleobase sequence of the primers, or component priming polymers of the amplification primer sets, can be selected to amplify the same or different target nucleic acid molecules of the sample such that one amplified molecule is shorter than at least one other amplified molecule. Changes in detectable signal attributable to the changes in the transfer of energy between the donor and/or acceptor moieties of each independently detectable primer complex can be determined. These results, for each different independently detectable primer complex, can be compared with the results of one or more control samples and/or with a standard curve to thereby determine or estimate the concentration of amplifiable nucleic acid.

Because independently detectable primer complexes can be used, these assays can be performed in multiplex mode in the same tube but individual amplification reactions, each with a single primer set, can also be performed in separate containers, vessels or tubes. In some embodiments, a portion of the sample is contacted with one of the at least two amplification primer sets in a separate container, vessel or tube. In some embodiments, each of the at least two amplification primer sets is contacted with a portion of the sample in a separate container, vessel or tube. In some embodiments, the assay is performed in a single tube on the sample, or a portion thereof.

In some embodiments, it is desirable to repeat the same assay more than one time so that average of the various assays can be used as a result. Thus, in some embodiments, the above described method can be performed on each of two or more portions of the same sample wherein the average or mean of the result obtained for each individual measurement is used to determine or estimate the state of degradation of the nucleic acid of the entire sample.

In some embodiments of the methods of the invention, the two or more primer sets can amplify the same target nucleic acid molecule. In another embodiment, the two or more primer sets can amplify different target nucleic acid molecules. In still other embodiments, certain of the primer sets can amplify the same target nucleic acid molecule whilst other primer sets amplify different target nucleic acid molecules.

In still some other embodiments, this invention pertains to a method for determining or estimating the concentration of nucleic acid in each of at least two portions of a sample. According to the method, each of two or more portions of a sample comprising nucleic acid is contacted with a different amplification primer set wherein at least one of the primers of each set is a primer complex. Each primer complex comprises at least one component priming polymer wherein the priming polymer comprises: 1) a priming segment that is complementary, or substantially complementary, to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site. Each primer complex further comprises at least one component-annealing polymer that, at a minimum, comprises one or more interacting groups wherein the interacting groups of the component polymers form and stabilize the primer complex. Each primer complex further comprises at least one set of donor and acceptor moieties wherein to each of at least two of the component polymers of each primer complex is linked at least one moiety from each set such that formation of the primer complex facilitates transfer of energy between donor and acceptor moieties of the set.

According to some embodiments, each sample portion can be contacted with at least the other reagents required to perform a nucleic acid amplification reaction. The nucleic acid amplification reaction can be performed on each sample portion wherein the two or more primer complexes can be used as primers and wherein, in the at least two sample portions, the nucleobase sequence of the primers, or component priming polymers of the amplification primer sets, can be selected to amplify the same or different target nucleic acid molecules of the sample such that, in one of the at least two sample portions, one amplified molecule is shorter than at least one other amplified molecule in another sample portion. Changes in detectable signal attributable to the changes in the transfer of energy between the donor and/or acceptor moieties of each primer complex can be determined. These results, for each primer complex, can be compared with one or more control samples and/or with a standard curve to thereby determine or estimate the state of degradation of the nucleic acid of the sample.

If the above-described method utilizes independently detectable primer sets, the result of more than one amplification reaction can be determined in a single tube. Accordingly, in some embodiments, a portion of the sample is contacted with one of the at least two amplification primer sets in a separate container, vessel or tube. In some embodiments, each of the at least two amplification primer sets is contacted with a portion of the sample in a separate container, vessel or tube.

In some embodiments, the above described method can be performed on repeats of each of two or more portions of the same sample wherein the average or mean of the result obtained for each individual measurement is used to determine or estimate the state of degradation of the nucleic acid of the entire sample.

There is no limit on the number of primer sets that can be used in performing the method. For example, 3, 4, 5, 6, 7, 8, 9 or 10 primer sets (or more) can be used to amplify the same or different target nucleic acid molecules. The greater the number of data points, the greater the confidence can be in the result. However, in multiplex assays, the number of primer sets may be limited by the number of independently detectable labels can be determined in a single assay.

In some embodiments of the methods of the invention, the amplified molecule or molecules can be substantially single stranded. Alternatively, the amplified molecule or molecules can be substantially double stranded. By “substantially”, we mean that ninety percent or more of the amplified nucleic acid is in the specified single stranded or double stranded form.

In some embodiments of the methods of the invention, the amplification process can be selected from the group consisting of Polymerase Chain Reaction (PCR), Ligase Chain Reaction (LCR), Strand Displacement Amplification (SDA), Transcription-Mediated Amplification (TMA), Q-beta replicase amplification (Q-beta) and Rolling Circle Amplification (RCA). For example, the amplification process can be asymmetric (See: Gyllensten et al., U.S. Pat. No. 5,066,584, incorporated herein by reference) or asynchronous PCR (See: Chen et al. WO01 /94638) where the amplified molecule is substantially single stranded. Where the amplified molecule or molecules are substantially double stranded, the amplification process can be conventional PCR. Whatever the amplification process chosen, if primer complex dissociation is to operate by indirect dissociation, the amplification can be designed so that the polymerase will copy back on the molecule created by polymerase extension of the primer complex (see discussion above).

In some embodiments of the methods of the invention, the shortest amplified molecule can be 295 or fewer nucleotides in length. In some embodiments, the longest amplified molecule can be 300 or more nucleotides in length. In some embodiments the shortest amplified molecule can be from about 50 to about 295 nucleotides in length and the longest amplified molecule can be from about 300 to about 2500 nucleotides in length.

In some embodiments of the methods of the invention, the operation of PCR clamping can be used. The process of PCR clamping is described in U.S. Pat. No. 5,891,625 and U.S. Pat. No. 5,972,610, herein incorporated by reference.

In some embodiments of the methods of the invention, the amount of nucleic acid in the sample can be estimated or determined by comparison to either one or more control samples and/or with respect to a standard curve. Typically the standard curve is prepared by extrapolation of the results of performing at least two, but typically more than two, control samples. Standard curves can be used where the assay parameters are well established and generate reproducible results.

Having described some embodiments of the invention, it will now become apparent to one of skill in the art that other embodiments incorporating the concepts described herein can be used. It is felt, therefore, that these embodiments should not be limited to disclosed embodiments but rather should be limited only by the spirit and scope of the following claims.

### EXAMPLES

### Example 1

### Time Course Study of DNA Degradation

Exposure of DNA to heat may result in DNA damages such as strand breaks, depurination, or deamination of cytosine. This damage to DNA templates can block Taq DNA polymerase during polymerase chain reaction amplification, resulting in a decrease in amplification products. In the following example, polymerase chain reaction (PCR) amplification was performed using primer complexes on heat-treated genomic DNA samples utilizing the primer sites separated by 1542 and 180 base pairs on the human ApoB gene. Control DNA samples, which have not been exposed to DNA damage, were also processed for purposes of amplification efficiency comparisons. The concentration of undamaged genomic templates valid for PCR amplification in the test sample can be quantified by comparing the yield of amplification product from the test sample against the yield of amplification product from control samples or with a standard curve. By comparing the relative changes in concentration of intact long and short DNA templates between samples, the level of DNA damage induced by heat can be assessed.

Test samples of Raji genomic DNA were incubated at 99° C. for various durations. These samples and control samples were subjected to PCR amplification using 200 nM of the VIC-labeled forward primer A or 67 nM of the 6-FAM-labeled forward primer B and the common reverse primer to generate the 1542-bp and 180-bp amplicons. In each PCR reaction vessel, the forward primers A and B were each combined with a common Q-PNA to thereby form a primer complex having a double stranded region and 3′ target-specific primer segment. Separate reactions for each primer complex were performed using 0.8 μM 13-mer Q-PNA, 1× Amplitaq Gold master mix, 2 units of Amplitaq Gold, using 200 ng heat-treated DNA samples in a 25 μl reaction volume. These reagents were obtained from Applied Biosystems in Foster City. Amplifications were carried out on ABI 7700 with initial enzyme activation at 95° C. for 10 minutes, followed by 34 cycles of PCR (95° C. for 30 seconds, 55° C. for 30 seconds, 72° C. minute) and extension at 72° C. for 10 minutes.

The following oligomers and primers were used in the example:

The forward primer A:

(Seq. ID No. 1)

5′(VIC)TCATTCGCAATCAGCTTGAAGGAATTCTTGAAAACGA

The forward primer B:

(Seq. ID No. 2)

5′(6-FAM)TCATTCGCAATCAATGATTTCCCTGACCTTGGC

The common reverse primer:

| | |
|---|---|
| 5′ACCTTAGGTGTCCTTCTAAGGATCCTG | (Seq. ID No. 3) |

Q-PNA:

| | |
|---|---|
| Ac-TGATTGCGAATGA(Dabcyl)-CONH₂ | (Seq. ID No. 4) |

The PCR reactions were examined for the characteristic fluorescent emissions using an ABI 7700. Fluorescence intensity for the VIC dye was measured at 555 nm and the 6-FAM dye was measured at 535 nm. The observed fluorescence was indicative of the level of the amplification product. For example, the 1542-bp amplicon in the test sample reactions was more significantly reduced over the course of the time than that of the 180-bp amplicon (Table I). Using dilution series of control samples processed in the same manner as the test sample, standard curves were generated to show a linear-log relationship between changes in fluorescence intensity and concentration of control sample amplicons. Standard curves were used to determine concentration of undamaged nucleic acid templates in the test samples with efficiency equivalent to the control sample template. The results indicated that the ratios of the concentrations of valid long vs. short DNA templates decreased as the heating time increased. Because the probability of damage to the nucleic acid is in proportion to length of the templates, use of this quantitative amplification assay facilitates the determination and/or estimation of nucleic acid degradation and/or nucleic acid concentration.

TABLE I

| | Changes in | | Calculated | | |
|---|---|---|---|---|---|
| Duration | Fluorescence intensity | | concentration of | | |
| of heat | Δ(post PCR-pre PCR) | | valid template (ng/μl) | | |
| treatment | VIC | FAM | 1542-bp | 180-bp | Ratio |
| 0 min. | 138 | 1367 | 8.12 | 7.99 | 1.02 |
| 2 min. | 92 | 1329 | 5.33 | 7.25 | 0.74 |
| 3 min. | 68 | 1389 | 4.28 | 8.45 | 0.51 |
| 4 min. | 41 | 1381 | 3.34 | 8.28 | 0.40 |
| 5 min. | 14 | 1333 | 2.61 | 7.33 | 0.36 |

References:
Lindahl, T. 1993. Instability and decay of the primary structure of the DNA. Nature 362: 709-715.
Eigner, J. E., Boldker, H. and Michaels, G. 1961. The thermal degradation of nucleic acids. Biochim. Biophys. Acta 51: 165-168.
Frederico, L. A., Kunkel, T. A. and Shaw, B. R. 1990. A sensitive genetic assay for the detection of cytosine deamination: determination of rate constants and the activation energy. Biochemistry 29: 2532-2537.
Pullinger, C. R., Hennessy, L. K., Chatterton, J. E., Liu, W., Love, J. A., Mendel, C. M., Frost, P. H., Malloy, M. J., Schumaker, V. N. and Kane, J. P. 1995. Familial ligand-defective apolipoprotein B. J. Clin. Invest. 95: 1225-1234.

1. A method comprising:
a) contacting a sample, or one or more portions thereof, comprising degraded nucleic acid with at least two amplification primer sets wherein at least one of the primers of each set is an independently detectable primer complex comprising:
i) at least one component priming polymer wherein the priming polymer comprises: 1) a priming segment that is complementary or substantially complementary to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site;
ii) at least one component annealing polymer that, at a minimum, comprises one or more interacting groups wherein the interacting groups of the component polymers form and stabilize the primer complex; and
iii) at least one independently detectable set of donor and acceptor moieties wherein to each of at least two of the component polymers of each independently detectable primer complex is linked at least one moiety from each set such that formation of the primer complex facilitates transfer of energy between donor and acceptor moieties of the set;
b) contacting the sample, or one or more portions thereof, with at least the other reagents required to perform a nucleic acid amplification reaction;
c) performing the nucleic acid amplification reaction on the sample, or one or more portions thereof, wherein the two or more primer complexes are used as primers and wherein the nucleobase sequence of the primers, or component priming polymers of the amplification primer sets, are selected to amplify the same or different target nucleic acid molecules such that one amplified molecule is shorter than at least one other amplified molecule;
d) determining changes in detectable signal attributable to the changes in the transfer of energy between the donor and/or acceptor moieties of each independently detectable primer complex; and
e) comparing the results of step (d) for each different independently detectable primer complex with one or more control samples and/or with a standard curve to thereby determine or estimate the state of degradation of the nucleic acid in the sample.

2. The method of claim 1, wherein the two or more primer sets amplify the same target nucleic acid molecule.

3. The method of claim 1, wherein the two or more primer sets amplify different target nucleic acid molecules.

4. The method of claim 1, wherein 3, 4, 5, 6, 7, 8, 9 or 10 primer sets are used to amplify the same or different target nucleic acid molecules.

5. The method of claim 1, wherein steps (a)-(e) are performed on each of two or more portions of the same sample and wherein the average or mean of the result obtained in each step (e) is used to determine or estimate the state of degradation of the nucleic acid of the sample.

6. The method of claim 1, wherein the amplified molecule or molecules are substantially single stranded.

7. The method of claim 1, wherein the amplified molecule or molecules are substantially double stranded.

8. The method of claim 6, wherein the amplification process is asymmetric or asynchronous PCR.

9. The method of claim 7, wherein the amplification process is PCR.

10. The method of claim 1, wherein the amplification process is selected from the group consisting of Polymerase Chain Reaction (PCR), Ligase Chain Reaction (LCR), Strand Displacement Amplification (SDA), Transcription-Mediated Amplification (TMA), Q-beta replicase amplification (Q-beta) and Rolling Circle Amplification (RCA).

11. The method of claim 1, wherein the shortest amplified molecule is 295 or fewer nucleotides in length and the longest amplified molecule is 300 or more nucleotides in length.

12. The method of claim 1, wherein the shortest amplified molecule is from about 50 to about 295 nucleotides in length and the longest amplified molecule is from about 300 to about 2500 nucleotides in length.

13. The method of claim 1, wherein all of the component polymers of each primer complex are nucleic acid polymers.

14. The method of claim 1, wherein at least one of the component polymers of each primer complex is a non-nucleic acid polymer.

15. The method of claim 14, wherein the component annealing polymer of each complex is a PNA oligomer.

16. The method of claim 15, wherein the component annealing polymer of each complex is the same PNA oligomer.

17. The method of claim 16, wherein the component annealing polymer is a C-terminal dabcyl labeled PNA oligomer.

18. The method of claim 1, wherein the component annealing polymer of each primer complex is a C-terminal dabcyl labeled PNA oligomer and the component priming polymer of each primer complex is a 5′-fluorophore labeled nucleic acid oligomer.

19. The method of claim 1, wherein the component annealing polymer of each primer complex is a C-terminal fluorophore labeled PNA oligomer and the component priming polymer of each primer complex is a 5′-quencher labeled nucleic acid oligomer.

20. The method of claim 1, wherein the each different independently detectable primer complex comprises an independently detectable donor fluorophore.

21. The method of claim 15, wherein PNA subunits of a component polymer have the formula: 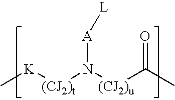
wherein,
each J is the same or different and is selected from the group consisting of:
H, R¹, OR¹, SR¹, NHR¹, NR¹₂, F, Cl, Br and I;
each K is the same or different and is selected from the group consisting of:
O, S, NH and NR¹;
each R¹ is the same or different and is an alkyl group having one to five carbon atoms which can optionally contain a heteroatom or a substituted or unsubstituted aryl group;
each A is selected from the group consisting of a single bond, a group of the formula; —(CJ₂)ₛ- and a group of the formula; —(CJ₂)ₛC(O)— wherein, J is defined above and each s is an integer from one to five;
each t is 1 or 2;
each u is 1 or 2; and
each L is the same or different and is independently selected from the group consisting of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine), other naturally occurring nucleobase analogs and other non-naturally occurring nucleobases.

22. The method of claim 1, wherein at least one component polymer of each primer complex is immobilized or tethered to a surface.

23. The method of claim 22, wherein the component priming polymer is immobilized or tethered to the surface.

24. The method of claim 1, further comprising the operation of PCR clamping.

25. The method of claim 1, wherein the assay is performed in the same tube.

26. The method of claim 1, wherein a portion of the sample is contacted with one of the at least two amplification primer sets in a separate container, vessel or tube.

27. The method of claim 1, wherein each of the at least two amplification primer sets is contacted with a portion of the sample in a separate container, vessel or tube.

28. A method comprising:
a) contacting a sample, or one or more portions thereof, comprising nucleic acid with at least two amplification primer sets wherein at least one of the primers of each set is an independently detectable primer complex comprising:
i) at least one component priming polymer wherein the priming polymer comprises: 1) a priming segment that is complementary or substantially complementary to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site;
ii) at least one component annealing polymer that, at a minimum, comprises one or more interacting groups wherein the interacting groups of the component polymers form and stabilize the primer complex; and
iii) at least one independently detectable set of donor and acceptor moieties wherein to each of at least two of the component polymers of each independently detectable primer complex is linked at least one moiety from each set such that formation of the primer complex facilitates transfer of energy between donor and acceptor moieties of the set;
b) contacting the sample, or one or more portions thereof, with at least the other reagents required to perform a nucleic acid amplification reaction;
c) performing the nucleic acid amplification reaction on the sample, or one or more portions thereof, wherein the two or more primer complexes are used as primers and wherein the nucleobase sequence of the primers, or component priming polymers of the amplification primer sets, are selected to amplify the same or different target nucleic acid molecules such that one amplified molecule is shorter than at least one other amplified molecule;
d) determining changes in detectable signal attributable to the changes in the transfer of energy between the donor and/or acceptor moieties of each independently detectable primer complex; and
e) comparing the results of step (d) for each different independently detectable primer complex with one or more control samples and/or with a standard curve to thereby determine or estimate the concentration of amplifiable nucleic acid in the sample.

29-54. (canceled)

55. A method comprising:
a) contacting each of at least two portions of a sample comprising degraded nucleic acid with a different amplification primer set wherein at least one of the primers of each primer set is a primer complex comprising:
i) at least one component priming polymer wherein the priming polymer comprises: 1) a priming segment that is complementary or substantially complementary to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site;
ii) at least one component annealing polymer that, at a minimum, comprises one or more interacting groups wherein the interacting groups of the component polymers form and stabilize the primer complex; and
iii) at least one set of donor and acceptor moieties wherein to each of at least two of the component polymers of each primer complex is linked at least one moiety from each set such that formation of the primer complex facilitates transfer of energy between donor and acceptor moieties of the set;
b) contacting each sample portion with at least the other reagents required to perform a nucleic acid amplification reaction;
c) performing the nucleic acid amplification reaction on each sample portion, wherein the two or more primer complexes are used as primers and wherein, in the at least two sample portions, the nucleobase sequence of the primers, or component priming polymers of the amplification primer sets, are selected to amplify the same or different target nucleic acid molecules of each sample portion such that, in one of the at least two sample portions, one amplified molecule is shorter than at least one other amplified molecule in another sample portion;
d) determining changes in detectable signal attributable to the changes in the transfer of energy between the donor and/or acceptor moieties of each primer complex; and
e) comparing the results of step (d) for each different primer complex with one or more control samples and/or with a standard curve to thereby determine or estimate the state of degradation of the nucleic acid in the sample.

56-79. (canceled)

80. A method comprising:
a) contacting each of at least two portions of a sample comprising nucleic acid with a different amplification primer set wherein at least one of the primers of each primer set is a primer complex comprising:
i) at least one component priming polymer wherein the priming polymer comprises: 1) a priming segment that is complementary or substantially complementary to a priming site within a target nucleic acid molecule; and 2) one or more interacting groups suitable for the formation of a complex with at least one other component polymer and wherein the primer complex is capable of performing as a primer in a nucleic acid amplification reaction upon hybridization of the priming segment to the priming site;
ii) at least one component annealing polymer that, at a minimum, comprises one or more interacting groups wherein the interacting groups of the component polymers form and stabilize the primer complex; and
iii) at least one set of donor and acceptor moieties wherein to each of at least two of the component polymers of each primer complex is linked at least one moiety from each set such that formation of the primer complex facilitates transfer of energy between donor and acceptor moieties of the set;
b) contacting each sample portion with at least the other reagents required to perform a nucleic acid amplification reaction;
c) performing the nucleic acid amplification reaction on each sample portion, wherein the two or more primer complexes are used as primers and wherein, in the at least two sample portions, the nucleobase sequence of the primers, or component priming polymers of the amplification primer sets, are selected to amplify the same or different target nucleic acid molecules of each sample portion such that, in one of the at least two sample portions, one amplified molecule is shorter than at least one other amplified molecule in another sample portion;
d) determining changes in detectable signal attributable to the changes in the transfer of energy between the donor and/or acceptor moieties of each primer complex; and
e) comparing the results of step (d) for each different primer complex with one or more control samples and/or with a standard curve to thereby determine or estimate the concentration of amplifiable nucleic acid in the sample.

81-104. (canceled)
